(19) United States
(12) Patent Application Publication
(10) Pub. No.: US 2006/0110359 A1
(43) Pub. Date: May 25, 2006

(54) Cellular delivery of natriuretic peptides

(76) Inventors: Juan Sanchez-Ramos, Tampa, FL (US); Shijie Song, Tampa, FL (US); Paul R. Sanberg, Spring Hill, FL (US); Siddharth G. Kamath, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/526,538

(22) PCT Filed: Sep. 8, 2003
(86) PCT No.: PCT/US03/28157

§ 371 Date: Oct. 11, 2005

Related U.S. Application Data
(60) Provisional application No. 60/319,530, filed on Sep. 6, 2002.

(51) Int. Cl. A61K 35/14; C12N 5/08

(52) U.S. Cl. 424/93.1; 435/366

(57) ABSTRACT

The present invention pertains to a method for treating deficits, such as neurological deficits, caused by focal or generalized edema associated with injury to the central nervous system, heart, liver, and kidney. The present invention further concerns a method for producing natriuretic peptides and pharmaceutical compositions comprising bone marrow stromal cells and effective amounts of retinoic acid and nerve growth factor to induce the bone marrow stromal cells to increase production of natriuretic peptides.

### CROSS-REFERENCE TO RELATED APPLICATION

The present application claims the benefit of priority of U.S. Provisional Application Ser. No. 60/319,530, filed Sep. 6, 2002, which is hereby incorporated by reference herein in its entirety, including any figures, tables, nucleic acid sequences, amino acid sequences, or drawings.

### BACKGROUND OF INVENTION

Brain natriuretic peptide (BNP) is a peptide of 26 residues, with a remarkable homology to atrial natriuretic peptide (ANP). The peptide exerts natriuretic-diuretic activity as well as a potent smooth muscle relaxant activity (Sudoh et al., Biochem Biophys Res Commun, 1988, 155:726-732). ANP administered into the rat lateral ventricle, or administered intravenously, has been reported to inhibit brain water and sodium accumulation associated with ischemic infarcts and in sub-arachnoid hemorrage-induced brain edema (Nakao et al., Neurosurgery, 1990, 27:39-43; discussion 43-34; Naruse et al., Acta Neurochir Suppl (Wien), 1990, 51:118-121; Doczi et al., Acta Neurochir (Wien), 1995, 132:87-91). ANP given after a 4-hour delay significantly reduced brain water and sodium 24 hours after an experimental brain hemorrhage in rats. Neither mannitol nor 8-bromo-cGMP affected brain edema. The anti-edematous effect of ANP has been attributed to inhibition of sodium transport and the coupled water influx.

Administration of BMSC intravenously or intracerebrally has been shown to result in significant improvement in the rate of recovery from the neurological deficits produced in a rat model of stroke (Li et al., Journal of Cerebral Blood Flow & Metabolism, 2000, 20:1311-1319; Chen et al., Stroke, 2001, 32:1005-1011; Li et al., Neurology, 2002, 59:514-523). Similar treatment with BMSC has also enhanced recovery from traumatic brain injury (Mahmood et al., Neurosurgery, 2001, 49:1196-1204; Mahmood et al., Journal of Neurosurgery, 2001, 94:589-595; Mahmood et al., J Neurotrauma, 2002, 19:1609-1617). In all these reports, the structural repair of the brain lesion does not correlate with the recovery, leading to the hypothesis that secretion of growth factors and cytokines mediate, in part, the enhanced recovery process. Indeed, secretion of growth factors such as brain derived neurotrophic factor (BDNF), nerve growth factor (NGF), vascular endothelial growth factor (VEGF), and hepatocyte growth factor (HGF) have been demonstrated in vitro and in vivo after transplantation of BMSC (Chen et al., J Neurosci Res, 2002, 69:687-691; Chen et al., Neuropathology, 2002a, 22:275-279; Li et al., Neurology, 2002, 59:514-523). Moreover, an immune-mediated response elicited by grafted bone marrow cells may play a role in recovery (Li et al., Neurology, 2002, 59:514-523).

The present inventors have discovered a new way to deliver anti-edema agents utilizing a cellular vehicle that generates natural anti-edemic agents, the natriuretic peptides. In light of the beneficial effects of intravenous administration or intracerebral grafting of BMSC in animal models of stroke and brain trauma, it is strongly suggested that the enhanced recovery from neurologic deficits is mediated in part by BNP secreted by BMSC in situ.

The natriuretic peptides have as one of their physiological functions the ability to increase excretion of sodium and water by the kidney and to pump sodium and water from individual cells. ANP are produced in great amounts in the heart, especially during heart failure. BNP is found in the hypothalamus of the brain, but is also found at much higher concentrations in heart tissue. These peptides have been shown to be effective when delivered directly into brain tissue (through an indwelling probe), but until the present inventors' discovery, these peptides could not be delivered systemically because of rapid proteolysis in the system circulation.

### BRIEF SUMMARY OF INVENTION

The present invention pertains to a method for treating deficits caused by focal or generalized edema associated with injury to organs or organ systems, such as the central nervous system, heart, liver, and kidneys. According to the method of the present invention, edema associated deficits are treated by the administration of cells that produce natriuretic peptides, such as bone marrow stromal cells. Where bone marrow stromal cells are utilized, the cells are preferably conditioned with retinoic acid and nerve growth factor before, during, or after administration to the patient, in order to increase the cells' production of natriuretic peptide. The method of the present invention can be used to treat neurological deficits of the central nervous system that result from stroke, trauma, toxins, and other nervous system insults.

In another aspect, the present invention concerns pharmaceutical compositions comprising bone marrow stromal cells and effective amounts of retinoic acid and nerve growth factor to induce the bone marrow stromal cells to increase production of natriuretic peptides.

In another aspect, the present invention provides a method for producing natriuretic peptides comprising culturing bone marrow stromal cells and isolating the natriuretic peptides from the bone marrow stromal cells. Preferably, the bone marrow stromal cells are cultured in the presence of retinoic acid and nerve growth factor, thereby inducing the bone marrow stromal cells to increase production of the natriuretic peptides.

In another aspect, the present invention pertains to cells that have been genetically modified to produce a natriuretic peptide.

### BRIEF DESCRIPTION OF DRAWINGS

FIG. 1 shows micrographs of bone marrow stromal cells (BMSCs) cultured in the presence or absence of growth factors. BMSCs were cultured under two conditions depicted in the rows A (Dulbecco Modified Eagle's Medium and fetal bovine serum (DMEM+FBS) for 6 days) and B (DMEM+FBS for 2 days, then retinoic acid and nerve growth factor (RA+NGF) for 4 days). Each row depicts the same visual field viewed with phase contrast microscopy (left frame), and fluorescence microscopy (middle and right frames). The column on the left reveals the morphological change from flat large epithelioid cells to fibroblastic morphology induced by adding RA+NGF (phase microscopy). The middle column shows BNP immunoreactive cells. The top row (condition A) illustrates several large cells (arrows) that do not show BNP immunoreactivity whereas all the fibroblastic cells in Condition B are BNP immunoreactive. The third column reveals DAPI-stained nuclei. (scale bar=50 μm).

FIG. 2 shows results from a single reverse transcriptase-polymerase chain reaction (PCR) experiment run to confirm the presence of brain natriuretic peptide in BMSCs. FIG. 2: Lanes 1, 3, and 5 were from cultures incubated in DMEM+FCS. Lanes 2, 4 and 6 were from cultures incubated in RA+NGF. Msh1 and nestin are normally expressed by neural progenitors and are also detected in the BMSC under both conditions of culture; BNP=brain natriuretic peptide; Msh1=musashi-1.

FIG. 3 shows results obtained by applying real-time PCR to RNA extracted from BMSC cultures. FIG. 3: RNA samples were extracted from each of four cultures prepared from two different donor sources. Conditions A and B were used in cells from one donor and conditions A and C were used in cells from a different donor. The RNA was reversed transcribed into cDNA and real-time PCR was carried out as described in the methods section. Amounts of human BNP and 18s RNAs were calculated using linear regression analysis from an external standard curve. Data is plotted as BNP mRNA −18s RNAs on the Y-axis and culture conditions on the X-axis.

FIG. 4 shows summary data using BMSCs from four different marrow samples. FIG. 4 2-way ANOVA demonstrated that culture conditions as well as cell/supernatant contributed significantly to the variance (p<0.05 for culture condition and p<0.001 for supernatant). t-tests with Bonferroni correction show significant differences between cells and supernatant for condition B* (p<0.001) and for condition C** (p<0.01) but not for condition A (p>0.05).

### BRIEF DESCRIPTION OF SEQUENCES

SEQ ID NO:1 is the gene encoding human brain natriuretic peptide (BNP) (NCBI ACCESSION # M31776; Seilhamer, J. J. et al., Biochem. Biophys. Res. Commun., 165(2):650-658, 1989):

| | |
|---|---|
| 1 | ctgtgagatc accccgtgct cccagcgctc acgtcggtcc tcggaaagcc ggggtcctcc |
| 61 | ctgccttttc cagcaacggt ggggtgggga ggcaggaaga aagcgccaac ctaggacccc |
| 121 | ggagatttgc agcaaaggaa gaagcgggag acgggcactt gtctgtgtct ccagcgcgtt |
| 181 | cctgcccccc gccgacccgg cccatttcta tacaaggtcg ctctgcccgg tctccacctc |
| 241 | ccacgtgcag gccgcggagg ggctcattcc cgggccctga tctcagaggc ccggaatgtg |
| 301 | gctgataaat cagagactag acctgcatgg caggcaggcc cgacactcag ctccaggata |
| 361 | aaaggccacg gtgtcccgag gagccaggag gagcaccccg caggctgagg gcaggtggga |
| 421 | agcaaacccg gacgcatcgc agcagcagca gcagcagcag aagcagcagc agcagcctcc |
| 481 | gcagtccctc cagagacatg gatccccaga cagcaccttc ccgggcgctc ctgctcctgc |
| 541 | tcttcttgca tctggctttc ctgggaggtc gttcccaccc gctgggcagc cccggttcag |
| 601 | cctcggactt ggaaacgtcc gggttacagg tgagagcgga gggcagctca gggggattgg |
| 661 | acagcagcaa tgaaagggtc ctcacctgct gtcccaagag gccctcatct ttcctttgga |
| 721 | attagtgata aaggaatcag aaaatggaga gactgggtgc cctgaccctg tacccaaggc |
| 781 | agtcggttca cttgggtgcc atgaagggct ggtgagccag gggtgggtcc ctgaggcttg |
| 841 | gacgccccca ttcattgcag gagcagcgca accatttgca gggcaaactg tcggagctgc |
| 901 | aggtggagca gacatccctg gagcccctcc aggagagccc ccgtcccaca ggtgtctgga |
| 961 | agtcccggga ggtagccacc gagggcatcc gtgggcaccg caaaatggtc ctctacaccc |
| 1021 | tgcgggcacc acgaagcccc aagatggtgc aagggtctgg ctgctttggg aggaagatgg |
| 1081 | accggatcag ctcctccagt ggcctgggct gcaaaggtaa gcaccccctg ccaccccggc |
| 1141 | cgccttcccc cattccagtg tgtgacactg ttagagtcac tttggggttt gttgtctctg |
| 1201 | ggaaccacac tctttgagaa aaggtcacct ggacatcgct tcctcttgtt aacagccttc |
| 1261 | agggccaagg ggtgcctttg tggaattagt aaatgtgggc ttatttcatt accatgccca |
| 1321 | caataccttc tccccacctc ctacttctta tcaaaggggc agaatctcct ttgggggtct |
| 1381 | gtttatcatt tggcagcccc ccagtggtgc agaaagagaa ccaaacattt cctcctggtt |
| 1441 | tcctctaaac tgtctatagt ctcaaaggca gagagcagga tcaccagagc aatgataatc |
| 1501 | cccaatttac agatgaggaa actgaggctc agagagttgc attaagcctc aaacgtctga |
| 1561 | tgactaacag ggtggtgggt ggcacacgat gaggtaagct cagcccctgc ctccatctcc |
| 1621 | caccctaacc atcatcaccc tctctctttc cctgacagtg ctgaggcggc attaagagga |
| 1681 | agtcctggct gcagacacct gcttctgatt ccacaagggg ctttttcctc aaccctgtgg |
| 1741 | ccgcctttga agtgactcat tttttttaat gtatttatgt atttatttga ttgttttata |
| 1801 | taagatggtt tcttaccttt gagcacaaaa tttccacggt gaaataaagt caacattata |
| 1861 | agctttatct tttgaaactg atttgtcttg gcgcattaaa aataatccct catttcaaag |
| 1921 | aa |

SEQ ID NO:2 is the amino acid sequence of human BNP:

MDPQTAPSRALLLLLFLHLAFLGGRSHPLGSPGSASDLETSGLQEQRNHL
QGKLSELQVEQTSLEPLQESPRPTGVWKSREVATEGIRGHRKMVLYTLRA
PRSPKMVQGSGCFGRKMDRISSSSGLGCKVLRRH.

SEQ ID NO:3 is the gene encoding human atrial natriuretic peptide (ANP) (NCBI ACCESSION # NM_{—}006172; Zivin, R. A. et al., Proc. Natl. Acad. Sci. USA, 81(20):6325-6329, 1984):

| | |
|---|---|
| 1 | tggcgaggga cagacgtagg ccaagagagg ggaaccagag aggaaccaga ggggagagac |
| 61 | agagcagcaa gcagtggatt gctccttgac gacgccagca tgagctcctt ctccaccacc |
| 121 | accgtgagct tcctcctttt actggcattc cagctcctag gtcagaccag agctaatccc |
| 181 | atgtacaatg ccgtgtccaa cgcagacctg atggatttca agaatttgct ggaccatttg |
| 241 | gaagaaaaga tgcctttaga agatgaggtc gtgcccccac aagtgctcag tgagccgaat |
| 301 | gaagaagcgg gggctgctct cagccccctc cctgaggtgc ctccctggac cggggaagtc |
| 361 | agcccagccc agagagatgg aggtgccctc gggcggggcc cctgggactc ctctgatcga |
| 421 | tctgccctcc taaaaagcaa gctgagggcg ctgctcactg cccctcggag cctgcggaga |
| 481 | tccagctgct tcgggggcag gatggacagg attggagccc agagcggact gggctgtaac |
| 541 | agcttccggt actgaagata acagccaggg aggacaagca gggctgggcc tagggacaga |
| 601 | ctgcaagagg ctcctgtccc ctggggtctc tgctgcattt gtgtcatctt gttgccatgg |
| 661 | agttgtgatc atcccatcta agctgcagct tcctgtcaac acttctcaca tcttatgcta |
| 721 | actgtagata aagtggtttg atggtgactt cctcgcctct cccaccccat gcattaaatt |
| 781 | ttaaggtaga acctcacctg ttactgaaag tggtttgaaa gtgaataaac ttcagcacca |
| 841 | tggac |

SEQ ID NO:4 is the amino acid sequence of human ANP:

MSSFSTTTVSFLLLLAFQLLGQTRANPMYNAVSNADLMDFKNLLDHLEEK
MPLEDEVVPPQVLSEPNEEAGAALSPLPEVPPWTGEVSPAQRDGGALGRG
PWDSSDRSALLKSKLRALLTAPRSLRRSSCFGGRMDRIGAQSGLGCNSFR
Y.

SEQ ID NO:5 is the gene encoding dog (Canine) brain natriuretic peptide (BNP) (NCBI ACCESSION # M31777; Seilhamer, J. J. et al., Biochem. Biophys. Res. Commun. 165(2):650-658, 1989):

| | |
|---|---|
| 1 | cgatcaggga tgttggggcg gaggaaacgg agggaaggag ggagcggagg aggcccgagg |
| 61 | actgttggtg tccccctcct gcccttttgg ggccaggccc acttctatac aaggcctgct |
| 121 | ctccagcctc caccccggcg ggtatggtgc aggcgcggag gggcgcattc ccccgccctg |
| 181 | agctcagcgg ccggaatgcg gccgataaat cagagataac cccaggcgcg ggataaggga |
| 241 | taaaaagccc ccgttgccgc gggatccagg agagcacccg cgccccaagc ggtgacactc |
| 301 | gaccccggtc gcagcgcagc agctcagcag ccggacgtct ctttccccac ttctctccag |
| 361 | cgacatggag ccctgcgcag cgctgccccg ggccctcctg ctcctcctgt tcttgcacct |
| 421 | gtcgccactc ggaggccgcc cccacccgct gggcggccgc agccccgcct cggaagcctc |
| 481 | ggaagcctca gaagcctcgg ggttgtgggc cgtgcaggtg agcgctcagc ctgcctgaag |
| 541 | gccgcggcgg gtggcagcag gtcacggggg cttagccact gtcccaagtc ctcagtctcc |
| 601 | cttgggaatt agtgataagg gaatcagaaa gtgacgagat tgggtgccag gactccatac |
| 661 | ccaaggcggc ggcttcactt gggtgcaagg gtggttccgc cccggcgtgg gttcctgagg |
| 721 | ctcaggccgt ccattgcagg agctgctggg ccgtctgaag gacgcagttt cagagctgca |
| 781 | ggcagagcag ttggccctgg aacccctgca ccggagccac agccccgcag aagccccgga |
| 841 | ggccggagga acgccccgtg gggtccttgc accccatgac agtgtcctcc aggccctgag |
| 901 | aagactacgc agccccaaga tgatgcacaa gtcagggtgc tttggccgga ggctggaccg |
| 961 | gatcggctcc ctcagtggcc tgggctgcaa tggtaagccg cctccctgcc gccttggctc |
| 1021 | cccctcccca gccccctggg ttcgaccctt ggaacccctt ctgggtttgt tgtctcgggg |
| 1081 | gatcacactc tgaggaaagg acatctggac atcgctcctt cttgctgaca gtcctaaggg |
| 1141 | ccaaggagta cgtttctgga aatactacgt gtggacatcg ttgtccaggg tccctaccca |
| 1201 | cctcctagcc ccctcctgcc tctcgcaccc aagggcagaa tcatcttagg atggaatcag |
| 1261 | tcgttgtctg gaagcatctc cttggagcag aaagagtcct aaacatcgtc ctcgtagctc |
| 1321 | tctctgtctg tctgtagcca cgaaggcaga ggtcagggtc accagggcag tgatgattcc |
| 1381 | cagttaacag aggaggagac tgaggtctag agagatggat tattccaaag cctcaaacat |
| 1441 | ccagatcggc tgagggtggg gttggtggca gggatggctc ctgggcttgg gaagctcgga |
| 1501 | tcctgcctca gtctcccacc tgacgccatc atccccctct ctctcctccc acagtgctga |
| 1561 | gaaagtatta aggaggaagt cccgactgcc cacatctgca ttggattctt cagcagcccc |
| 1621 | tgagcccctt ggaagcagat cttatttatt cgtatttatt tatttattta tttcgattgt |
| 1681 | tttatataag atgatcctga cgcccgagca cggattttcc acggtgaaat aaagtcaacc |
| 1741 | ttagagcttc ttttgaaacc gatttgtccc tgtgcattaa aagtaacaca tcatttaaaa |
| 1801 | aaa |

SEQ ID NO:6 is the amino acid sequence of dog (Canine) BNP:

MEPCAALPRALLLLLFLHLSPLGGRPHPLGGRSPASEASEASEASGLWAV
QELLGRLKDAVSELQAEQLALEPLHRSHSPAEAPEAGGTPRGVLAPHDSV
LQALRRLRSPKMMHKSGCFGRRLDRIGSLSGLGCNVLRKY.

SEQ ID NO:7 is the hBNP-exon M2 probe.
SEQ ID NO:8 is a forward primer for human BNP.
SEQ ID NO:9 is a reverse primer for human BNP.

SEQ ID NO:10 is the nucleotide sequence encoding human BNP (NCBI ACCESSION # M31776):

| | |
|---|---|
| 1 | atggatcccc agacagcacc ttcccgggcg ctcctgctcc tgctcttctt gcatctggct |
| 61 | ttcctgggag gtcgttccca cccgctgggc agccccggtt cagcctcgga cttggaaacg |
| 121 | tccgggttac aggagcagcg caaccatttg cagggcaaac tgtcggagct gcaggtggag |
| 181 | cagacatccc tggagcccct ccaggagagc ccccgtccca caggtgtctg gaagtcccgg |
| 241 | gaggtagcca ccgagggcat ccgtgggcac cgcaaaatgg tcctctacac cctgcgggca |
| 301 | ccacgaagcc ccaagatggt gcaagggtct ggctgctttg ggaggaagat ggaccggatc |
| 361 | agctcctcca gtggcctggg ctgcaaagtg ctgaggcggc attaa |

SEQ ID NO:11 is the nucleotide sequence encoding human ANP (NCBI ACCESSION # NM_{—}006172):

| | |
|---|---|
| 1 | atgagctcct tctccaccac caccgtgagc ttcctccttt tactggcatt ccagctccta |
| 61 | ggtcagacca gagctaatcc catgtacaat gccgtgtcca acgcagacct gatggatttc |
| 121 | aagaatttgc tggaccattt ggaagaaaag atgcctttag aagatgaggt cgtgccccca |
| 181 | caagtgctca gtgagccgaa tgaagaagcg ggggctgctc tcagccccct ccctgaggtg |
| 241 | cctccctgga ccggggaagt cagcccagcc cagagagatg gaggtgccct cgggcggggc |
| 301 | ccctgggact cctctgatcg atctgccctc ctaaaaagca agctgagggc gctgctcact |
| 361 | gcccctcgga gcctgcggag atccagctgc ttcgggggca ggatggacag gattggagcc |
| 421 | cagagcggac tgggctgtaa cagcttccgg tactga |

SEQ ID NO:12 is the mouse type-B natriuretic peptide gene (NCBI ACCESSION # S58667; Steinhelper, M. E., Circ. Res. 72(5): 984-992, 1993; coding sequences (CDS)=join nucleotides 211-336, 531-753, 1188-1204):

| | |
|---|---|
| 1 | gaattctcag gtcctgagct cagccggcag gaatgcagct gataaatcag agataacccc |
| 61 | acccctactc cgtgaaaagg tctggccgga cactcagccc cagtataaaa ggcagaggca |
| 121 | ccgttgttga agacaccagt gcacaagctg cttggggagg cgagacaagg gagaacacgg |
| 181 | catcattgcc tggcccatcg cttctgcggc atggatctcc tgaaggtgct gtcccagatg |
| 241 | attctgtttc tgcttttcct ttatctgtca ccgctgggag gtcactccca tcctctggga |
| 301 | agtcctagcc agtctccaga gcaattcaag atgcaggtga gcactgaggg tctgcctgaa |
| 361 | gggtttggga agcggcaatg aaaagacctc gagtcctttg ggaattagcc atgtgagagt |
| 421 | cagcaaagtg aaagattggg cagcatatct cttaactgat gagcactatg gaaggatggg |
| 481 | ggattcaggt gtgtgtgttt ctgacgtctg ggctccccaa tccatcacag aagctgctgg |
| 541 | agctgataag agaaaagtca gaggaaatgg cccagagaca gctcttgaag gaccaaggcc |
| 601 | tcacaaaaga acacctaaaa agagtccttc ggtctcaagg cagcaccctc cgggtccagc |
| 661 | agagacctca aaattccaag gtgacacata tctcaagctg ctttgggcac aagatagacc |
| 721 | ggatcggatc cgtcagtcgt ttgggctgta acggtgagca cctaccttgc cacttccctg |
| 781 | caaagctgca ccacccatcc catccccgtg catgctaccc ttagaggccc ctaggtttgc |
| 841 | tatctggcat actcctgcag cctgtcagga aatatcacat gggttctgca ttacattctc |
| 901 | acaggtcagc acctaccttc catcagaggg tcacacgctc tgaggagcag actgctgatg |
| 961 | tctatcaccc cttcacaagg cagaaagagt ctgagcattc ccctcaggca aagggcatgc |
| 1021 | ccaacccact ttacaggaga aacagaggcc ctctgagata gctttttcca gagccttaaa |
| 1081 | cttcgacatc atctggggac tgaagatggg ggtgtggtgg tggtggggga ctcggcacct |
| 1141 | gcttcagttt cacttcgagt gtgacattgc ctgtctctcc tccacagcac tgaagttgtt |
| 1201 | gtaggaagac ctccttggct gcaggagagc tccagtttct gactctgccg ggtctctttc |
| 1261 | cctagctctg gaccacctct gaagtgatcc tatttattta tttatttata tttattattt |
| 1321 | atttttattt ttatttttta atttaatttt gttgtttttc acagctgttg ttacttggag |
| 1381 | cacaaactgc cacaacataa taaacatacg ttatttcctg cttttgaaaa gg |

SEQ ID NO:13 is the mouse BNP gene for natriuretic peptide (NCBI ACCESSION # D16497; Ogawa, Y. et al., J. Clin. Invest., 93(5):1911-1921, 1994; coding sequences (CDS): join nucleotides 210-335, 530-752, 1196-1212):

| | |
|---|---|
| 1 | gaattctcag gtcctgagct cagccggcag gaatcagctg ataaatcaga gataacccca |
| 61 | cccctactcc gtgaaaaggt ctggccggac actcagcccc agtataaaag gcagaggcac |
| 121 | cgttgttgaa gacaccagtg cacaagctgc ttggggaggc gagacaaggg agaacacggc |
| 181 | atcattgcct ggcccatcgc ttctgcggca tggatctcct gaaggtgctg tcccagatga |
| 241 | ttctgtttct gcttttcctt tatctgtcac cgctgggagg tcactcctat cctctgggaa |
| 301 | gtcctagcca gtctccagag caattcaaga tgcaggtgag cactgagggt ctgcctgaag |
| 361 | ggtttgggaa gcggcaatga aaagacctcg agtcctttgg gaattagcca tgtgagagtc |
| 421 | agcaaactga aagattgggc agcatatctc ttaactgatg agcactatgg aaggatgggg |
| 481 | gattcaggtg tgtgtgtttc tgacgtctgg gctccccaat ccatcacaga agctgctgga |
| 541 | gctgataaga gaaaagtcgg aggaaatggc ccagagacag ctcttgaagg accaaggcct |
| 601 | cacaaaagaa cacccaaaaa gagtccttcg gtctcaaggc agcaccctcc gggtccagca |
| 661 | gagacctcaa aattccaagg tgacacatat ctcaagctgc tttgggcaca agatagaccg |
| 721 | gatcggatcc gtcagtcgtt tgggctgtaa cggtgagcac ctaccttgcc acttccctgc |
| 781 | aaagctgcac acccatccca tccccgtgca tgctaccctt agaggcccct aggtttgcta |
| 841 | tctggcatac tcctgcagcc tgtcaggaaa tatcacatgg gttctgcatt acattctcac |
| 901 | aggtcagcac ctaccttcca tcagaggggt cacacgctct gagggagcag actgcctgat |
| 961 | gtctaatcac cccttcacaa ggcagaaaga gttctgagca tttcccctca ggcaaagggc |
| 1021 | atgcccaacc cactttacag gagaaacaga ggccctgtga gatagctttt tccagagcct |
| 1081 | taaacttcga catcatctgg ggactgaaga tgggggtgtg gtggtggtgg gggactcggc |
| 1141 | acctgcttca gtttcacttc cgagtgtgac attgccctgt ctctcctccc cacagcactg |
| 1201 | aagttgttgt aggaagacct cctggctgca ggagactcca gtttctgact ctgcctgggt |
| 1261 | ctctttcccc agctctggga ccacctttga agtgatccta tttatttatt tatttatatt |
| 1321 | tatttttatt tttatttttt aatttatttt gttgtttttc tacaagactg tttcttatct |
| 1381 | tggagcacaa acttgccaca acataataaa catagcgtat ttcctgcttt tgaaaaggat |
| 1441 | ttgtgtccgt gagtttcaat ctatctct |

SEQ ID NO:14 is the rat BNP mRNA (NCBI ACCESSION # M25297; Kojima M. et al., Biochem. Biophys. Res. Commun., 159(3):1420-1426, 1989; coding sequence (CDS): join nucleotides 58-423):

| | |
|---|---|
| 1 | gcgagacaag agagagcagg acaccatcgc agctgcctgg cccatcactt ctgcagcatg |
| 61 | gatctccaga aggtgctgcc ccagatgatt ctgctcctgc ttttccttaa tctgtcgccg |
| 121 | ctgggaggtc actcccatcc cctgggaagt cctagccagt ctccagaaca atccacgatg |
| 181 | cagaagctgc tggagctgat aagagaaaag tcagaggaaa tggctcagag acagctctca |
| 241 | aaggaccaag gccctacaaa agaacttcta aaaagagtcc ttaggtctca agacagcgcc |
| 301 | ttccggatcc aggagagact tcgaaattcc aagatggcac atagttcaag ctgctttggg |
| 361 | cagaagatag accggatcgg cgcagtcagt cgcttgggct gtgacgggct gaggttgttt |
| 421 | taggaagacc tcctggctgc agactccggc ttctgactct gcctgcggct cttctttccc |
| 481 | cagctctggg accacctctc aagtgatcct gtttatttat ttgtttattt atttattttt |
| 541 | atgttgctga ttttctacaa gactgtttct tatcttccag cacaaacttg ccacagtgta |
| 601 | ataaacatag cctatttctt gcttttgg |

SEQ ID NO:15 is the porcine BNP mRNA (NCBI ACCESSION # M25547; Porter et al., J. Biol. Chem., 264(12):6689-6692, 1989; coding sequence (CDS): join nucleotides 94-216, 463-718, 1273-1289):

| | |
|---|---|
| 1 | caggctgcta ggaagtgaaa agtgaacctg gacccagctc agcggcagca gcagcggcag |
| 61 | caggcagcag cctctatcct ctcctccagc cacatgggcc cccggatggc gcttccccgc |
| 121 | gtgctcctgc tcctgttctt gcacctgttg ctgctaggat gccgttccca tccactgggt |
| 181 | ggcgctggcc tggcctcaga actgccaggg atacaggtga gccctgatga actgcttaga |
| 241 | cttggttggc tgggagggcg cggacagcag caactaacgg gtccccacct actgttccaa |
| 301 | gagggctcta acctcctttg ggaactagtg ataaggggtt agaaggcagc caggctgggg |
| 361 | gtgaggaccc cgctcccaag gcagttggtt cgcttcagca ccatcaagag tgatgggtcc |
| 421 | aggtgcgagt tcctgaggct cgggctcccc cacccatccc aggagctgct ggaccgcctg |
| 481 | cgagacaggg tctccgagct gcaggcggag cggacggacc tggagcccct ccggcaggac |
| 541 | cgtggcctca cagaagcctg ggaggcgagg gaagcagccc ccacgggggt tcttgggccc |
| 601 | cgcagtagca tcttccaagt cctccgggga atacgcagcc ccaagacgat gcgtgactct |
| 661 | ggctgctttg ggcggaggct ggaccggatc ggctccctca gcggcctggg ctgcaatggt |
| 721 | gagcacccac ccccattccc actgcacgcc ccggttagca tcacttctgg gtttgatgtc |
| 781 | tctggggacc aaactccgag aaaaggacac ctggatatca ctctttcttg ttgccagtcc |
| 841 | tcaaggccaa ggagcgcctt cctggaaaaa ttaaatttgg acagcattca ctagcatgac |
| 901 | tatgagtccc cacccacctt ctcgccaccc cctgcctctc tcacccaagg cggcagaatt |
| 961 | actttaggat gtaaattctg tcattgcctg gctgccgctc ctgggagcaa aaagagaact |
| 1021 | aaacctcttc cccctggttt cccctcaact gtctgtggct gcaaaggcag agggcaggat |
| 1081 | caccagggtg atgacaagtc ccagcttaca aggaggaaac tcaggtccag agagatggat |
| 1141 | tatcccaaag ccccaaacat ccagttctgc tgaagaaggc gggtggcagg ggtggcacgt |
| 1201 | ggtgggggga agcccaggtc ctgcctgcct ctcaccctaa tgtcatcctc accctctctc |
| 1261 | tcccccccac agtgctcagg aggtactgag aagtcctggc tgacaacctc tgtgtccgct |
| 1321 | tctccaacgc ccctcccctg ctccccttca aagcaactcc tgtttttatt tatgtattta |
| 1381 | tttatttatt tatttggtgg ttgtatataa gacggttctt atttgtgagc acattttttc |
| 1441 | catggtgaaa taaagtcaac attagagctc tgtcttttg |

SEQ ID NO:16 is the bovine ANP gene (NCBI ACCESSION # M13145; Vlasuk, G. P. et al., Biochem. Biophys. Res. Commun., 136(1):396-403, 1986; coding sequence (CDS): join nucleotides 313-432, 531-857, 1383-1394):

| | |
|---|---|
| 1 | ctgcagctga gggtcctggg ggttgtcggg gctgctcaag gcagaggggc tgtgacaagc |
| 61 | aggctggact gataacttta aaagggcatc ttctgctgct tcctcactca gctgctttat |
| 121 | cactgcaagt gacagaatgg ggagggttcc gtccctctcc cggacgagct cccagagagc |
| 181 | cagggggcta taaaaagagg aggctcaggg cagctgggag acagagacgg acaaaggcca |
| 241 | acagcaaaag gccaaagagg acagggagga ggcagcaagc accagaccga ccattccttg |
| 301 | accgacgcca gcatgggctc ctccgccatc accgtgagct tcctcctctt tctggcattt |
| 361 | cagctcccag ggcaaacagg agcaaatccc gtgtatggct ctgtgtccaa tgcagacctg |
| 421 | atggatttca aggtagggcc agggaacggc gatggtctgg ggctgagggg gttgtgacat |
| 481 | tgtgccaggc gagcgagacc tctccctttc cctgttttcc ttttgtaaag aatttgctgg |
| 541 | accgtttgga ggacaagatg cctttagaag atgaggctgt gccctcacaa gtactaagtg |
| 601 | agcagaatga agaagctggg gcccctctca gccccctttc agagatgcct ccctggatgg |
| 661 | gggaggtcaa cccagcccag agagaggggg gcgtcctcgg gcggggcccc tgggaatcct |
| 721 | ccgatagatc tgccctcctg aagagcaagc tgagggcact gctcactgcc cctcggagcc |
| 781 | tgcggaggtc cagctgcttc gggggaagga tggacaggat tggagcccag agtggattgg |
| 841 | gctgcaacag cttccgggta agaggacctg agaatggaaa tgggatgggg aggaaggaaa |
| 901 | ttgtggcttc attgaagttc aaaccttgtg aaagaacatc gccagggaat gccttcagta |
| 961 | ggaaagggac agcatagaag caaccccttt gaaatttctg ccccaacttg gcagggagga |
| 1021 | gggtgtgctc tgagtctcag gacaatgata ccaacctagc tacagttttc tgagagaatg |
| 1081 | ctaagaaaaa aagactttac tgccacgagc actggggact taaattgttc atggggccaa |
| 1141 | ataacctgtg ctttgctgat tggtagtttg tgtcctttgc agaatcatca gatcccaaag |
| 1201 | gattgaaatt gagcaggact gactttacta gttcctaatg ggcaatttgt ttaccagttt |
| 1261 | atagaagtca gagggtcatc aggctggagt ggaggctggt gggaagggag cacagtctga |
| 1321 | tgaagctggc ttttccagtg gagtcaggtc accaaaccaa acatgtctct gctctcttgt |
| 1381 | agtatcgaag ataatggcca gggaggaaaa ggcaggccag gccctgggca gtcttcaaga |
| 1441 | gaatcccctg gggtctctca ctcaactttg tcgcatctgg ttgccatcaa gttgagctgt |
| 1501 | gaccgagcat tcaagcatca gcttcttgtc aacatttctc acattttatg ctaaatgtag |
| 1561 | acaaagtgat ttaactgtgg ccttctccac ctctcccacc catgtgttaa gttttaatca |
| 1621 | cctgttacca acatcagttt gaaaatgaat aaacttcagc accatggaca gaagcagtag |
| 1681 | gctcgggttg gtgtgatttc tttcatttcc ggaagggagt tcagcctgat actccttgtc |
| 1741 | attttacctt ttgttggaga gaagaattc |

SEQ ID NO:17 is the rat iso-ANP gene (NCBI ACCESSION # M60731; Roy, R. N. and Flynn, T. G., Biochem. Biophys. Res. Commun., 171(1):416-423, 1990; coding sequence (CDS): join nucleotides 58-183, 405-627, 1080-1096):

| | |
|---|---|
| 1 | gcgagacaag agagagcagg acaccatcgc agctgcctgg cccatcactt ctgcagcatg |
| 61 | gatctccaga aggtgctgcc ccagatgatt ctgctcctgg ttttccttaa tctgtcgccg |
| 121 | ctgggaggtc actcccatcc cctgggaagt cctagccagt ctccagaaca atccacgatg |
| 181 | caggtgagca ccgagggtct gcatagggag atggagcctg cctgaagggt tttgggcagc |
| 241 | agcaatgaaa agacctcatg tcctttggga attaaccacg cgagagtcag gaaacggaaa |
| 301 | gattgggcag cagatccctt aaccacaggc actgtggaag ggtgggggag ccaggtgtgt |
| 361 | atgtgtgtgt gtgtctgagg tctgggcttc ccaattcgtc acagaagctg ctggagctga |
| 421 | taagagaaaa gtcagaggaa atggctcaga gacagctctc aaaggaccaa ggccctacaa |
| 481 | aagaacttct aaaaagagtc cttaggtctc aagacagcgc cttccggatc caggagagac |
| 541 | ttcgaaattc caagatggca catagttcaa gctgctttgg gcagaagata gaccggatcg |
| 601 | gcgcagtcag tcgcttgggc tgtgacggtg agcacctacc ttgccgcttc cctgcaaagc |
| 661 | tgcacgcatc ccgttcccct gcatgccgcc ctcagaggcc ccttggtttg ctctcagaca |
| 721 | tacttgcaca gcctgcctct accttaccga cagtcttcaa gaccaaggca gtctgtcagg |
| 781 | aagtctcaca tgggtacttc attacaccgt cccaggtgag cacctacctc cttcagaggt |
| 841 | gtcacaggct tcccagggaa cagactgcct gatgtctgat cactctgagc atctcccctc |
| 901 | cgtcttcacc aaactgaatt atccgaggca aagggcaggc ccagtgagat agcttttccc |
| 961 | agagccgtta aacttcgaca tcatctggga accaaagatg ggggtgcggt gtggcagggg |
| 1021 | aagctcagct cctgcctcag tttcactccc cagtctgaca ctggtttctc ctcccacagg |
| 1081 | gctgaggttg ttttaggaag acctcctggc tgcagactcc ggcttctgac tctgcctgcg |
| 1141 | gctcttcttt ccccagctct gggaccacct ctcaagtgat cctgtttatt tatttgttta |
| 1201 | tttatttatt tttatgttgc tgattttcta caagactgtt tcttatcttc cagcacaaac |
| 1261 | ttgccacagt gtaataaaca tagcctattt cttgcttttg g |

SEQ ID NO:18 is the amino acid sequence of bovine natriuretic peptide:

1 mgssaitvsf llflafqlpg qtganpvygs vsnadlmdfk nlldrledkm pledeavpsq

61 vlseqneeag aplsplsemp pwmgevnpaq reggvlgrgp wessdrsall ksklrallta

121 prslrrsscf ggrmdrigaq sglgcnsfry rr.

SEQ ID NO:19 is the amino acid sequence of mouse BNP:

1 mdllkylsqm ilfflflyls plgghsyplg spsqspeqfk mqkllelire kseemaqrql

61 lkdqgltkeh pkrvlrsqgs tlrvqqrpqn skvthisscf ghkidrigsv srlgcnalkl

121 l

SEQ ID NO: 20 is the amino acid sequence of rat BNP:

1 mdlqkylpqm illllflnls plgghshplg spsqspeqst mqkllelire kseemaqrql

61 skdqgptkel lkrvlrsqds afriqerlrn skmahssscf gqkidrigav srlgcdglrl

121 f.

SEQ ID NO:21 is the amino acid sequence of porcine BNP:

1 mgprmalprv llllflhlll lgcrshplgg aglaselpgi qelldrlrdr vselqaertd

61 leplrqdrgl teaweareaa ptgvlgprss ifqvlrgirs pktmrdsgcf grrldrigsl

121 sglgcnylrr y.

### DETAILED DISCLOSURE OF INVENTION

The present invention is based at least partly on the discovery that brain natriuretic peptides (BNP) and atrial natriuretic peptides (ANP) are produced in high levels by human bone marrow stromal cells (BMSCs) in vitro.

The present invention pertains to a method for treating deficits caused by focal or generalized edema associated with injury to organs or organ systems, such as the central nervous system, heart, liver and kidney. According to the method of the present invention, edema associated deficits are treated by the administration of cells that produce natriuretic peptides, such as BMSCs. Where BMSCs are utilized, the cells are preferably conditioned with retinoic acid and nerve growth factor before, during, or after administration to the patient, in order to increase the cells' production of natriuretic peptide. However, conditioning of BMSCs with retinoic acid and nerve growth factor is not necessary since BMSCs produce BNP in vivo.

The natriuretic peptides delivered by the transplanted cells increase excretion of sodium and water within the patient, mimicking the effects of direct infusion of the peptides. For example, the method of the present invention can be used to treat neurological deficits of the central nervous system that result from stroke, trauma, toxins, and other nervous system insults. Administration of natriuretic peptide producing cells into the brain results in decreased brain edema at the penumbra zone of injury, facilitating a more rapid recovery. Thus, infusion of natriuretic peptide producing cells, such as BMSC, systemically or intracerebrally is a novel way to treat any acute injury to brain, spinal cord or peripheral organs that results in edema.

ANPs do not cross the blood-brain-barrier, but BMSCs infused systemically do penetrate preferentially at the zone of the infarct or injury. BMSCs have also been shown to localize at the ischemic heart following systemic intravenous administration.

In another aspect, the present invention concerns pharmaceutical compositions comprising bone marrow stromal cells and effective amounts of retinoic acid and nerve growth factor to induce the bone marrow stromal cells to increase production of natriuretic peptides. The pharmaceutical composition can be adapted for various forms of parenteral administration, such as intravenous or intracerebral routes. Administration can be continuous or at distinct intervals as can be determined by a person skilled in the art.

The pharmaceutical compositions of the subject invention can be formulated according to known methods for preparing pharmaceutically useful compositions. Formulations are described in a number of sources which are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In another aspect, the present invention provides a method for producing natriuretic peptides comprising culturing bone marrow stromal cells and isolating the natriuretic peptides from the bone marrow stromal cells. Peptide isolation methods known in the art can be utilized. For example, antibodies specific for natriuretic peptide can be used to isolate the peptides from culture. Preferably, the bone marrow stromal cells are cultured in the presence of retinoic acid and nerve growth factor, thereby inducing the bone marrow stromal cells to increase production of the natriuretic peptides by the bone marrow stromal cells. According to one embodiment of the present invention, bone marrow stromal cells are conditioned for about two to about four days in vitro with retinoic acid (0.5 μM) and NGF (100 ng/μL). After the cultures reach confluency, the cells can be lifted by incubation with trypsin (0.25%) and 1 mM EDTA at 37° C. for 3-4 min. The BMSC cells are suspended in phosphate buffered saline (PBS) and infused into the systemic circulation (total of about 1-3 million cells).

A wide variety of media, salts, media supplements, and products for media formulation can be utilized to culture the BMSCs in the presence of retinoic acid and nerve growth factor, thereby conditioning the cells to increase production of natriuretic peptides according to the method of the present invention. Examples of these substances include, but are not limited to, carrier and transport proteins (e.g., albumin), biological detergents (e.g., to protect cells from shear forces and mechanical injury), biological buffers, growth factors, hormones, hydrosylates, lipids (e.g., cholesterol), lipid carriers, essential and non-essential amino acids, vitamins, sera (e.g., bovine, equine, human, chicken, goat, porcine, rabbit, sheep), serum replacements, antibiotics, antimycotics, and attachment factors. These substances can be present in various classic and/or commercially available media, which can also be utilized with the subject invention. Examples of such media include, but are not limited to, Ames' Medium, Basal Medium Eagle (BME), Click's Medium, Dulbecco's Modified Eagle's Medium (DMEM), DMEM/Nutrient Mixture F12 Ham, Fischer's Medium, Minimum Essential Medium Eagle (MEM), Nutrient Mixtures (Ham's), Waymouth Medium, and William's Medium E.

According to methods of the present invention, natriuretic peptide producing cells can be administered as cell therapy to alleviate the symptoms of a wide variety of disease states and pathological conditions associated with edema, in various stages of pathological development. Treatment with natriuretic peptide producing cells is intended to include prophylactic intervention to prevent onset of the symptoms associated with the particular edema-associated deficit, as well as intervention to alleviate or eliminate symptoms of an edema-associated deficit that are being presented by the patient at the time of administration of the cells. For example, natriuretic peptide producing cells can be used to treat acute disorders (e.g., stroke or myocardial infarction), and administered acutely, subacutely, or in the chronic state. Similarly, the natriuretic peptide producing cells can be used to treat chronic disorders (e.g., Parkinson's disease), and administered preventatively and/or prophylactically, early in the disease state, in moderate disease states, or in severe disease states.

The following pathological conditions are examples of diseases or conditions that cause edemic injury which may be treated in accordance with the present invention: cerebrovascular accidents (also referred to as stroke), brain trauma, and spinal cord injuries. The following are examples of deficits associated with or induced by edemic injury: paralysis, loss of sensation, inability to speak or understand language, loss of consciousness and coma.

Preferably, treatment will substantially correct an edema-induced deficit. However, that may not always be possible. Thus, treatment according to the present invention, and with the cells and pharmaceutical compositions of the invention, may lead to improvement in function without complete correction.

The number of cells to be used will vary depending on the nature and extent of the edemic tissue. Typically, the number of cells used in transplantation will be in the range of about one hundred thousand to several million. One or more transplants can be carried out to further improve function.

Methods for transplantation of cells into human and non-human mammals are known to those skilled in the art and described in the scientific literature. The terms “transplantation”, “administration”, and “implantation” are used herein interchangeably and include the transplantation of cells which have been grown in vitro, and may have been genetically modified to produce natriuretic peptides, as well as the transplantation of material extracted from another organism. Natriuretic peptide producing cells can be transplanted by means of microsyringe infusion of a known quantity of cells in the target area. The phrase “intracerebral transplantation” as used herein includes transplantation into any portion of the brain, and is not restricted to the front and/or larger portion of the brain.

The natriuretic peptide producing cells can be administered as autografts, syngeneic grafts, allografts, and xenografts, for example. As used herein, the term “graft” refers to one or more cells intended for implantation within a human or other animal. Hence, the graft can be a cellular or tissue graft, for example.

The natriuretic peptide producing cells can be administered in an open manner to the target anatomical site, as in the heart during open heart surgery, or in the brain during stereotactic surgery, or by systemic procedures such as intravascular interventional methods using catheters going to the blood supply of the specific organs, or by other interventional methods known in the art.

Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. As used herein, the term “patient” is intended to include such human and non-human mammalian species. The cells used in the subject invention can be any human or non-human mammalian cells.

Combinations of cell types can be co-administered to enhance therapeutic potential. For example, a trophic factor-producing cell line can be co-administered with a neuronal cell line that has been genetically modified to produce a natriuretic peptide, such as BNP or ANP. Sertoli cells can be co-administered with natriuretic peptide producing cells of a species different from that of the recipient, such that the Sertoli cells provide local immunosuppression of the xenograft.

Methods for transplanting various nerve tissues as allografts and xenografts have been described previously (Freeman T. B. et al., Progress in Brain Research, 1988, Chapter 61, Elsevier Science Publishers, 78:473-477; Freeman T. B. et al, Parkinson's Disease: Advances in Neurology, 2001, Chapter 46, Lippincott Williams & Wilkins, 86:435-445; Freeman T. B. et al., Annals of Neurology, 1995, 38(3):379-387; Freeman T. B. et al., Progress in Brain Research, 2000, Chapter 18, Elsevier, Amsterdam, 127:405-411; Olanow C. W. et al. The Basal Ganglia and New Surgical Approaches for Parkinson's Disease, Advances in Neurology, 1997, 74:249-269; Bjorklund et al., Neural Grafting in the Mammalian CNS, 1985, p. 709, Elsevier, Amsterdam; Das G. D., “Neural Grafting in the Mammalian CNS, 1985, Chapter 3, p. 23-30, Elsevier, Amsterdam). These procedures include intraparenchymal transplantation, i.e., within the host brain tissue (as compared to outside the brain or extraparenchymal transplantation) achieved by injection or deposition of tissue within the host brain so as to be opposed to the brain parenchyma at the time of transplantation.

Methods for intraparenchymal transplantation include, for example: (i) injecting the donor cells within the host brain parenchyma (e.g., stereotactically, using image guidance, and/or with a catheter attached to a pump, such as a MEDTRONIC system); and (ii) preparing a cavity by surgical means to expose the host brain parenchyma and then depositing the graft into the cavity. Such methods provide parenchymal apposition between the graft and host brain tissue at the time of grafting, and both facilitate anatomical integration between the graft and host brain tissue.

Alternatively, the graft can be placed into the cerebral spinal fluid (CSF), either by open surgical injection, intraventricularly via a needle or ventricular reservoir, into the lumbar subarachnoid space using a lumbar puncture, or into any CSF site using a pump and a catheter (e.g., MEDTRONIC). These methods would lend themselves to repeated administration over time, to the CSF or to the brain. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain can be made by drilling a hole and piercing the dura to permit the needle of a microsyringe to be inserted. The microsyringe can be mounted in a stereotactic frame and three-dimensional stereotactic coordinates are selected for placing the needle into the desired location of the brain or spinal cord. Image guidance methods can also be utilized. The cells of the subject invention can also be introduced into the putamen, caudate nucleus, pallidum, nucleus basalis, hippocampus, cortex, cerebellum, subcortical white matter, other regions of the brain, as well as the spinal cord.

The methods of the subject invention also contemplate the administration of cells that have been genetically modified to produce a natriuretic peptide, such as BNP or ANP. Such genetically modified cells can be administered alone or in combinations with different types of cells. Thus, genetically modified cells of the invention can be co-administered with other cells, which can include genetically modified cells or non-genetically modified cells. Genetically modified cells may serve to support the survival and function of the co-administered cells, for example.

SEQ ID NOs. 1 and 3 are genes encoding human BNP and human ANP, respectively. SEQ ID NOs. 10 and 11 are the coding sequences of human BNP and human ANP, respectively. In specific embodiments, the cells of the invention are genetically modified with nucleotide sequences encoding human BNP and/or human ANP (SEQ ID NOs. 2 and 4, respectively), such as the nucleotide sequences of SEQ ID NO: 1, 3, 10, or 11. In other embodiments, nucleotide sequences encoding non-human mammalian homologs of natriuretic peptides are used, such as nucleotide sequences encoding SEQ ID NO:6, SEQ ID NO:18, SEQ ID NO:19, SEQ ID NO:20, and SEQ ID NO:21. Exemplified nucleotide sequences encoding non-human mammalian homologs of natriuretic peptides are SEQ ID NO:5, SEQ ID NO:12, SEQ ID NO:13, SEQ ID NO:14, SEQ ID NO:15, SEQ ID NO:16, and SEQ ID NO:17.

The term “genetic modification” as used herein refers to the stable or transient alteration of the genotype of a cell of the subject invention by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides in addition to those encoding natriuretic peptides. A translation initiation codon can be inserted as necessary, making methionine the first amino acid in the sequence. The term “genetic modification” is not intended to include naturally occurring alterations such as that which occurs through natural viral activity, natural genetic recombination, or the like. The genetic modification may confer the ability to produce natriuretic peptides wherein the cell did not previously have the capability, or the modification may increase the amount of natriuretic peptides produced by the cell, e.g., through increased expression.

Exogenous nucleic acids can be introduced into a cell by viral vectors (retrovirus, modified herpes virus, herpes virus, adenovirus, adeno-associated virus, and the like) or direct DNA transfection (lipofection, calcium phosphate transfection, DEAE-dextran, electroporation, and the like), for example.

Preferably, the exogenous nucleotide sequence encoding a natriuretic peptide is operably linked to a promoter sequence that permits expression of the nucleotide sequence in a desired tissue within the patient. The promoters can be inducible or tissue specific as necessary. Means for inducible regulation of human BNP are known (Quan H. et al., Am. J. Physiol. Heart Circ. Physiol., 2001, 280:H368-H376). Examples of other promoters that can be utilized to express nucleotides encoding natriuretic peptides within a mammalian cell, such as a human cell, include cytomegalovirus promoter (Boshart et al., Cell, 1985, 41:521-530) and SV40 promotor (Subramani et al., Mol. Cell. Biol., 1981, 1:854-864).

The term “operably-linked” is used herein to refer to an arrangement of flanking sequences wherein the flanking sequences so described are configured or assembled so as to perform their usual function. Thus, a flanking sequence operably-linked to a coding sequence may be capable of effecting the replication, transcription and/or translation of the coding sequence. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a natriuretic peptide will typically have its own operably-linked promoter sequence.

The nucleotide sequences encoding natriuretic peptides used in the subject invention include “homologous” or “modified” nucleotide sequences. Modified nucleic acid sequences will be understood to mean any nucleotide sequence obtained by mutagenesis according to techniques well known to persons skilled in the art, and exhibiting modifications in relation to the normal sequences. For example, mutations in the regulatory and/or promoter sequences for the expression of a polypeptide that result in a modification of the level of expression of a polypeptide according to the invention provide for a “modified nucleotide sequence”. Likewise, substitutions, deletions, or additions of nucleic acid to the polynucleotides of the invention provide for “homologous” or “modified” nucleotide sequences. In various embodiments, “homologous” or “modified” nucleic acid sequences have substantially the same biological or serological activity as the native (naturally occurring) natriuretic peptide. A “homologous” or “modified” nucleotide sequence will also be understood to mean a splice variant of the polynucleotides of the instant invention or any nucleotide sequence encoding a “modified polypeptide” as defined below.

A homologous nucleotide sequence, for the purposes of the present invention, encompasses a nucleotide sequence having a percentage identity with the bases of the nucleotide sequences of between at least (or at least about) 20.00% to 99.99% (inclusive). The aforementioned range of percent identity is to be taken as including, and providing written description and support for, any fractional percentage, in intervals of 0.01%, between 20.00% and 99.99%. These percentages are purely statistical and differences between two nucleic acid sequences can be distributed randomly and over the entire sequence length.

In various embodiments, homologous sequences exhibiting a percentage identity with the bases of the nucleotide sequences of the present invention can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polynucleotide sequences of the instant invention. Homologous nucleotide and amino acid sequences include, but are not limited to, mammalian homologues of the human natriuretic peptide sequences. Homologous nucleotide sequences, when expressed, exhibit substantially the same biological activity (e.g., anti-edema effects) as the native sequence, as can be determined in vitro or in vivo.

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman Proc. Natl. Acad. Sci. USA, 1988, 85(8):2444-2448; Altschul et al. J. Mol. Biol., 1990, 215(3):403-410; Thompson et al. Nucleic Acids Res., 1994, 22(2):4673-4680; Higgins et al. Methods Enzymol., 1996, 266:383-402; Altschul et al. J. Mol. Biol., 1990, 215(3):403-410; Altschul et al. Nature Genetics, 1993, 3:266-272).

In another embodiment, the natriuretic peptide producing cells are derived from transgenic animals, and thus are in a sense already genetically modified. There are several methods presently used for generating transgenic animals. A typical technique is direct microinjection of DNA into single-celled fertilized eggs. Other methods include retro-viral-mediated transfer, or gene transfer in embryonic stem cells. These techniques and others are detailed by Hogan et al. in Manipulating the Mouse Embryo, A Laboratory Manual (Cold Spring Harbor Laboratory Ed., 1986).

In addition to the objective of delivering natriuretic peptides to a patient in need thereof, the genetically modified cells of the subject invention can be administered to a patient for cell/gene therapy, e.g., for in vivo delivery of various biomolecules, such as the trophic factors described above. Alternatively, the genetically modified cells can be used as biological “factories” to provide the product of the exogenous DNA (e.g., natriuretic peptide) and/or the natural product of the modified cells in vitro, or in vivo within an animal.

Genetically modified cells can be stem cells or non-stem cells. Thus, non-stem cells (e.g., specialized or mature cells, and their precursor or progenitor cells) can be genetically modified to produce natriuretic peptides. Genetic modification with nucleotide sequences encoding natriuretic peptides, such as BNP and ANP, can be used to produce cells that have therapeutic potential for injuries related to multiple or specific organs. For example, cardiomyocytes or their progenitors can be genetically modified to target heart injury; hepatocytes or their progenitors can be genetically modified to target liver injury; and islet cells or their progenitors can be genetically modified to target diabetes. Any of the over 200 cells naturally occurring in mammals can be genetically modified to provide cellular delivery of one or more natriuretic peptides. Examples of cells that can be genetically modified include, but are not limited to, bone marrow cells, stem cells, neural cells, Sertoli cells, fat cells, placental cells, fibroblasts, and umbilical cord blood cells.

According to the methods of the present invention, whether genetically modified or non-genetically modified, the natriuretic peptide producing cells can be co-administered with other therapeutic agents useful in treating defects, trauma, or diseases, such as growth factors, antibiotics, or neurotransmitters.

Cells can be genetically modified to include regulators, inducible promoters, tissue-specific promoters, on-off genes, or suicide genes. Genetically modified cell lines can include more than one genetic construct.

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al., Blood, 1996, 87:3822.)

### Materials and Methods

Human Bone Marrow Stromal Cell Cultures. Human bone marrow cells are not easy to obtain for basic research since they are a scarce resource for treatment of certain leukemias. We used an easily accessible alternative source of bone marrow stromal cells (BMSC). The method has been described in detail (Song, S. and Sanchez-Ramos, J., “Preparation of Neural Progenitors from Bone Marrow and Umbilical Cord Blood” in Protocols for Neural Stem Cell Methods, Zigova, T. et al., Eds., 2002, pp. 79-88). Bone marrow from volunteer donors were routinely passed through a nylon filter to remove debris from the sample prior to preparation for bone marrow replacement. The nylon filter retains bony chips with adherent bone marrow cells, fatty tissue and other debris. The nylon filters were washed with sterile saline solution five times 4 of 19 and centrifuged to remove bone chips. The cells in the supernatant were then processed as described and plated in uncoated polyethylene culture flasks in Dulbecco's minimal essential medium (DMEM; Gibco/BRL, Inc) and fetal bovine serum 10% (FBS; Hyclone). When adherent cells reached 60-70% confluence, they were raised from the plates, resuspended and re-plated in new flasks.

Five human bone marrow donors provided the nylon filters that contain bone marrow debris from which BMSC were subsequently prepared.

1. Male 34 y/o BMSC; assays performed at passage 3 and passage 5

2. Male 50 y/o BMSC; assays performed at passage 1 and passage 3

3. Male 59 y/o BMSC; assays performed at passage 1 and passage 3

4. Female 44 y/o BMSC; assays performed at passage 11 and passage 13

5. Female 34 y/0 BMSC: immunocytochemical assay at passage 4.

The cultures were incubated under three distinct media conditions. Condition A: DMEM+FBS10% for 6 days. Condition B: DMEM+FBS10% for two days followed by Nerve Growth Factor (NGF 100 ng/mL)+all-trans retinoic acid (RA; 0.5 μg/mL) for four days. Condition C: N2+20 ng/mL of Epidermal Growth Factor (EGF; GIBCO)+10 ng/mL of basic Fibroblast Growth Factor (bFGF; GIBCO) for two days followed by NGF+RA for four days.

Immunocytochemistry and estimates of cell density. Cells from a 34 y/o female were plated in 35 mm multi-well plates at passage four with a density of approximately 7.5×10⁵ cells per well. After a total of six days in vitro (under conditions described above), the cultures were fixed in 5% phosphate-buffered paraformaldehyde for 30 minutes, washed three times with phosphate buffered saline and incubated with rabbit antiserum to human BNP-32 (PENINSULA LABORATORIES, Inc.; Belmont, Calif.) at a dilution of 1:200 for 24 hrs at 4° C. FITC conjugated anti-rabbit antibodies (CHEMICON INTERNATIONAL Inc.) were used to visualize the BNP immunoreactive cells. Cultures were also incubated without primary antibody to control for non-specific staining by the anti-rabbit antibody, DAPI (4′,6-diamidino-2-phenylindole, dihydrochloride; MOLECULAR PROBES, Eugene Oreg.) staining of nuclei was used to visualize total number of cells per visual field. Estimates of cell density were based on counts of DAPI-stained nuclei in 10 visual fields per culture dish.

DNA Microarray Analysis. Total RNA was isolated from cell cultures using the RNA STAT-60 kit according to the protocol recommended by the manufacturer (TEL-TEST “B,” Inc. Friendswood, Tex. 77546). Following RNA isolation, its OD density was measured at 260 nm, and stored at 280° C. Integrity was tested on 1% nondenaturing Seakem LE agarose gel (FMC BIO-PRODUCTS, Rockland, Me.). As previously described the samples were probed on the human genome U95A array (HGU95A) from AFFYMETRIX, Inc (Sanchez-Ramos et al., 2001). The single array represents; 12,500 sequences. The experiment was done at the Functional Genomics Core, Microarray Facility, at the H. Lee Moffitt Cancer Center and Research Institute using GeneChip Fluidics station 400, a Gene-Chip Hybridization oven, and an HP GeneArray scanner. Analysis of the GeneChip microarray hybridization pattern was performed using GeneChip Analysis Suite 4.0 software.

RT-PCR. Both RT and PCR were performed with PERKIN ELMER's GENEAMP RNA PCR kit. RT was performed using total RNA (1 μg) and random hexamers as a primer. PCR was done using Human BNP (GeneBank accn.# M31776) primers (Forward: nt. 1320-1341 and Reverse: nt. 1597-1578) on a PE 9700 thermocycler (PERKIN ELMER, Foster City, Calif.).

Real Time Quantitative RT-PCR. Real-time quantitative RT-PCR analyses were performed using the ABI PRISM 7700 Sequence Detection System (APPLIED BIOSYSTEMS, Foster City, Calif.). Primers and probes for human BNP were designed and synthesized by APPLIED BIOSYSTEMS (Foster City, Calif.) from GenBank accession number M31776. The probe hBNP-exonM2 (5′-CGCTGCTCCTGTAAC-3′) (SEQ ID NO:7) was labeled with 6-carboxyfluorescein as the reporter and a minor groove binder moiety (MGB) on the 3′-end. The forward primer was 5′-GCCTCGGACTTGGAAACGT-3′ (SEQ ID NO:8) and the reverse primer was 5′-TGCAGCTCCGACAGTTTGC-3′ (SEQ ID NO:9).

Sample RNA (5 μl of 50 ng/μl, 5 μl of 400 ng/μl) was transcribed into cDNA using Superscript II-reverse transcriptase (LIFE TECHNOLOGIES, Inc.) and 250 ng of random hexamers (LIFE TECHNOLOGIES, Inc.) following the manufacturer's directions in a final volume of 20 μl. For the construction of standard curves RNA was diluted in DEPC-treated water (AMBION, Austin, Tex.) before reverse transcription.

PCR was carried out with the TAQMAN Universal PCR Master Mix (APPLIED BIOSYSTEMS) using 3 μl of diluted cDNA, 200 nM probe, and 900 nM in a 30-μl final reaction mixture. After a 2-min incubation at 50° C., AMPLITAQ GOLD was activated by a 10-min incubation at 95° C. Each of the 40 PCR cycles consisted of 15 s of denaturation at 95° C. and hybridization of probe and primers for 1 min at 60° C. Amounts of human BNP and 18s RNAs were calculated using linear regression analysis from an external standard curve.

Radioimmunoassay for Brain Natriuretic Peptide (BNP). The assay is based upon the competition of ¹²⁵I-BNP and BNP of the sample binding to the limited quantity of specific antibodies for BNP. Cells and media were diluted with 100% ethanol (1:2 dilution), vortexed, and allowed to stand at 4° C. for 30 minutes. The samples were centrifuged and the supernatants were air-dried and stored at −80° C. until time of assay. The samples were incubated with rabbit anti-BNP antibody and normal rabbit serum for 24 hours at 4° C. Then, the samples were incubated with ¹²⁵I-BNP (13,000 cpm) for 24 hours at 4° C. Goat antirabbit IgG serum were added to the samples and incubated for 2 hours at room temperature. The samples were centrifuged at 3000 g for 20 minutes at 4° C. The radioactivity was counted with a gamma-counter. A standard curve was constructed by measuring the amount of ¹²⁵I-BNP bound to known concentrations of BNP.

Following is an example which illustrates materials, methods, and procedures for practicing the invention. The example is illustrative and should not be construed as limiting.

### EXAMPLE

### Production of Brain Natriuretic Peptide (BNP) by Bone Marrow

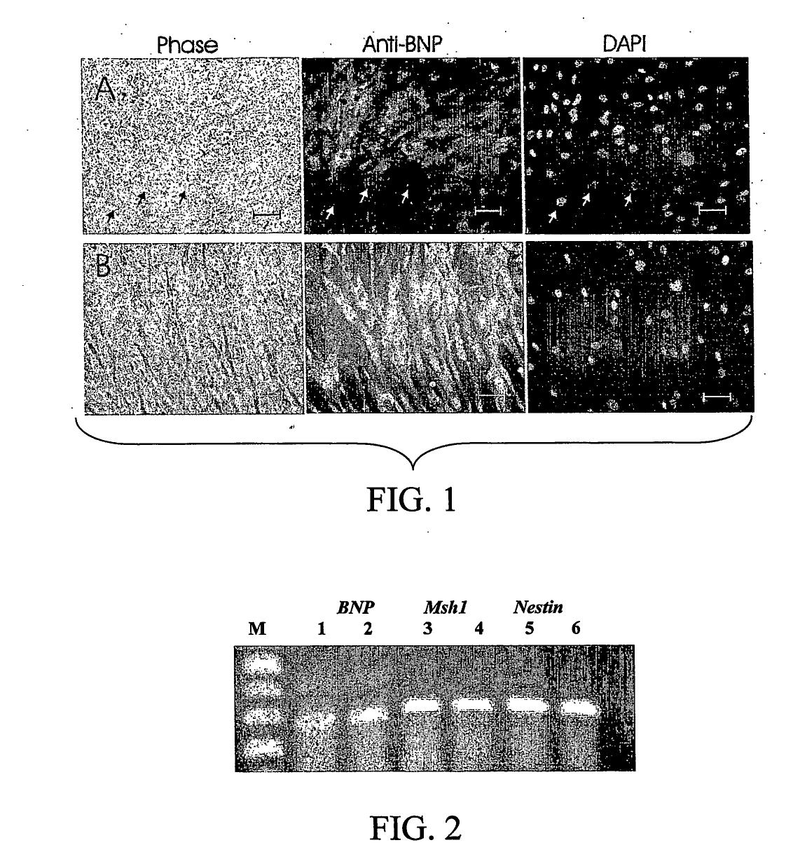

BMSC cultures were incubated with a) DMEM and FCS 10% for six days or with b) DMEM and FCS 10% for two days with a change of medium to retinoic acid and NGF for four days or with c) N2+EGF+bFGF for two days followed by NGF+RA for four days. Examination of the cultures under phase contrast microscopy revealed a change in morphology when growth factors were added (See FIG. 1). The untreated cultures (condition A) remained predominantly flat and assumed a sheet-like arrangement of cells (FIG. 1, row A). The cultures treated with growth factors conditions B or C (FIG. 1, row B) assumed a fibroblastic morphology. Under fluorescence microscopy all sets of cultures were immunoreactive for BNP. However, in condition A (no RA or growth factors), a small number of large flat cells were not BNP immunoreactive. In cultures treated with growth factors (FIG. 1, row B), most of the fibroblastic cells were immunoreactive for BNP. The mean density of cells in the untreated cultures (estimated by counting DAPI-stained nuclei) was 6.2×10⁵ cells per culture dish (with a range of 4.0 to 7.5×10⁵); the mean density of cells in the growth factor treated cultures (condition B) was 4.5×10⁵ cells per culture dish (range of 3.2 to 5.4×10⁶). Although there was a slight trend towards decreased numbers of cells as they changed morphology following the addition of growth factors, the difference in cell density under the two cell culture conditions after six days in vitro was not significant. The studies of BNP protein expression were carried out after six days in vitro. The cell densities were approximately 6.2×10⁵ in condition A and 4.5×10⁵ in conditions B and C (RA and growth factor treated cultures). For studies of gene expression using RT-PCR, cultures were grown in T-75 culture flasks at cell densities of approximately 3×10⁶ cells per flask. In a screening study of gene expression by BMSC undergoing differentiation, total RNA was extracted, and cDNA was prepared for hybrization with the oligonucleotides contained on the human U95A array (AFFYMETRIX, Inc.). Preliminary evidence revealed that transcripts for BNP were highly upregulated in the RA+NGF treated cultures in two microarray experiments using two different donor samples. Since this was designed to be quick survey of the transcriptional profile of these cultures, there were insufficient samples and experiments to perform statistical or cluster analyses. Review of the microarray data for changes in ANP and other regulators of sodium homeostasis, including rennin and aldosterone, was performed and none of these regulators were increased in RA-induced BMSC differentiation.

To validate the presence of BNP mRNA in the cell cultures, RT-PCR and quantitative RT-PCR was performed. In addition, a sensitive radioimmunoassay method was employed to quantify the levels of BNP protein produced within the cells and secreted into the medium.

FIG. 2 shows results from a single RT-PCR experiment run to confirm the presence of BNP in the BMSCs. The band in the RA+NGF column suggests a greater intensity than in the DMEM control sample, though this example was not designed to be quantitative. Also shown in the same gel are bands for Musashi-1 and nestin mRNA, both of which are considered to be markers of neural progenitors (Kaneko, Y. et al., Developmental Neuroscience, 2000, 22:139-153; Lendahl, U. et al., Cell, 1990, 60:585-595). There have been a number of investigations that have reported neural protein markers expressed by bone marrow stromal cells, but it remains uncertain as to whether these cells differentiate into functional neurons (Sanchez-Ramos, J. R. J. Neurosci. Res., 2002, 69:880-893).

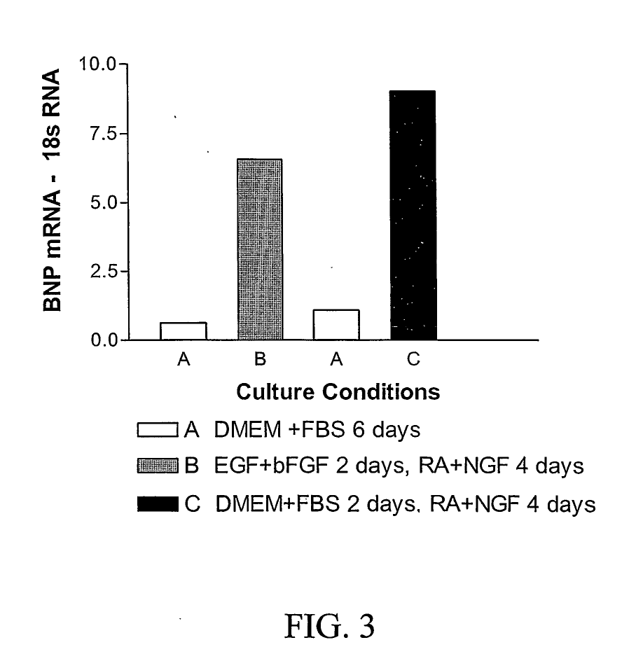

FIG. 3 shows the results obtained by applying real-time PCR to the RNA extracted from the cultures. Quantitative data were derived from two experiments using BMSC samples from different donors. BNP mRNA was increased 8 fold under condition B (RA+NGF) and 11 fold under condition C (EGF, bFGF, RA, NGF) compared to condition A (no growth factors). Insufficient number of samples were analyzed to perform a statistical analysis. In order to determine whether expression of BNP protein was increased, and to gather sufficient samples to perform statistical analyses, a sensitive radioimmunoassay was applied to 8 samples of BMSC incubated under three different conditions.

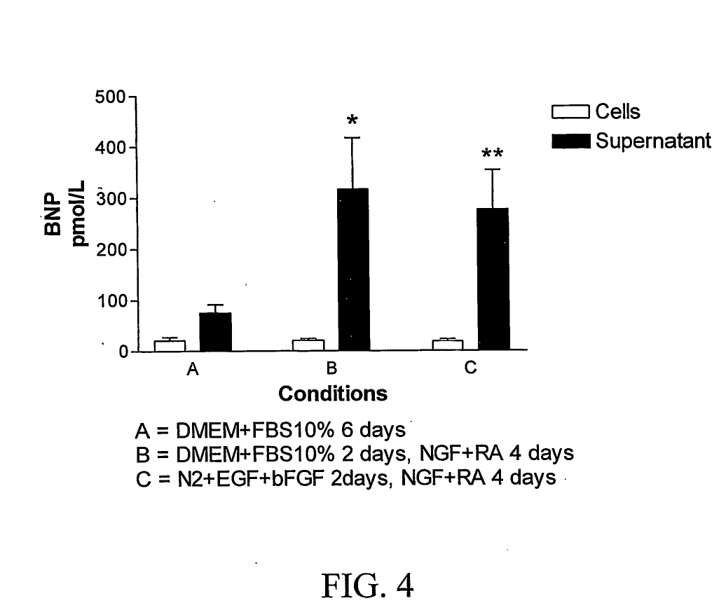

FIG. 4 shows summary data using BMSC from 4 different marrow samples (donated by subjects 1 to 4). BMSC from each of the donors was assayed at two different passages (n=8). BNP levels were measured in both the cells and supernatant in each of the three culture conditions. The levels of BNP measured within the cells did not change for any of the culture conditions and remained around 20 pmol/L. However, the mean levels of BNP released into the media was significantly increased (34 fold) by addition of retinoic acid and growth factors (p<0.001 in condition B and p<0.01 in condition C). The mean supernatant BNP level was 75 (SD=16, n=8) in cultures incubated with DMEM+FCS for 6 days (Condition A). In cultures incubated for 2 days with DMEM+FBS followed by 4 days with RA+NGF (Condition B), the mean BNP level was 317 (SD=100, n=8). When cultures were incubated for 2 days with EGF+FGF followed by 4 days with RA+NGF (Condition C), the mean levels of BNP were 276 (SD=77, n=8). 2-way ANOVA demonstrated that culture conditions as well as cell/supernatant contributed significantly to the variance (p<0.05 for culture condition and p<0.001 for supernatant). t-tests with Bonferroni correction show significant differences between cells and supernatant for condition B (p<0.001) and for condition C (p<0.01) but not for condition A (p>0.05). Even though the number of cells per dish in growth factor-treated cultures tended to be lower than DMEM+FBS treated cultures after 6 days in vitro, the average concentration of BNP released into the medium by the cultures incubated with RA and growth factors was significantly higher.

This is the first report that bone marrow is capable of producing significant levels of BNP. The mean levels of BNP secreted by BMSC with and without addition of growth factors (mean of 75 pmol/L in DMEM and 317 pmol/L in growth factor-treated cultures) far exceeds the BNP found circulating in the serum and CSF of normal human subjects. The mean serum BNP level in 124 healthy subjects was 1.8+/−1.0 pmol/L (SD), with a range of 0.6-5.5 pmol/L (Jensen et al., Scand J Clin Lab Invest, 1997, 57:529-540). In eight patients with congestive heart failure, the median BNP level was 30.5 pmol/L, with a range of 3.9-65.3. The mean BNP concentration in human cerebrospinal fluid collected from fifteen patients without neurological disorders has been reported to be 0.27+/−0.10, pmol/L (mean+/−S.D (Kaneko et al., Brain Res, 1993, 612:104-109).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A method of treating a deficit in a patient caused by focal or generalized edema, said method comprising administering an effective amount of cells that produce a natriuretic peptide to a patient in need thereof.

2. The method of claim 1, wherein the cells are bone marrow stromal cells.

3. The method of claim 2, wherein the bone marrow stromal cells have been conditioned with retinoic acid and nerve growth factor before, during, or after said administering.

4. The method of claim 1, wherein the cells produce at least one natriuretic peptide selected from the group consisting of brain natriuretic peptide and atrial natriuretic peptide.

5. The method of claim 1, wherein the cells are administered to the patient intravenously.

6. The method of claim 1, wherein the deficit is a neurological deficit caused by focal or generalized edema of the central nervous system.

7. The method of claim 1, wherein the deficit is selected from the group consisting of paralysis, loss of sensation, inability to speak, inability to understand known language, loss of consciousness, and coma.

8. The method of claim 1, wherein the deficit is caused by focal or generalized edema at a site selected from the group consisting of brain, spinal cord, heart, liver, and kidney.

9. The method of claim 1, wherein the focal or generalized edema is caused by a pathological condition selected from the group consisting of cerebrovascular accident, brain trauma, and spinal cord injury.

10. The method of claim 1, wherein the cells have been genetically modified to produce the natriuretic peptide.

11. The method of claim 10, wherein the cells have been genetically modified with a nucleotide sequence encoding a natriuretic peptide and an operably linked promoter sequence.

12. The method of claim 11, wherein the nucleotide sequence comprises a sequence selected from the group consisting of SEQ ID NO:1, SEQ ID NO:3, SEQ ID NO:5, SEQ ID NO:10, SEQ ID NO:11, SEQ ID NO:12, SEQ ID NO:13, SEQ ID NO:14, SEQ ID NO:15, SEQ ID NO:16, and SEQ ID NO:17 or a mammalian homolog of any of the foregoing.

13. The method of claim 1, wherein the natriuretic peptide is selected from the group consisting of SEQ ID NO:2, SEQ ID NO:4, SEQ ID NO:6, SEQ ID NO:18, SEQ ID NO:19, SEQ ID NO:20, and SEQ ID NO:21, or a mammalian homolog of any of the foregoing.

14. A method for delivering a natriuretic peptide to a target anatomical site comprising administering a bone marrow stromal cell to the target anatomical site.

15. The method of claim 14, wherein the bone marrow stromal cell has been conditioned with retinoic acid and nerve growth factor prior to said administering.

16. A method for producing natriuretic peptides comprising culturing bone marrow stromal cells and isolating the natriuretic peptides from the bone marrow stromal cells.

17. The method of claim 16, wherein said culturing is carried out in the presence of retinoic acid and nerve growth factor, thereby inducing the bone marrow stromal cells to increase production of the natriuretic peptides by the bone marrow stromal cells.

18. A pharmaceutical composition comprising bone marrow stromal cells and a pharmaceutically acceptable carrier.

19. The pharmaceutical composition of claim 18, wherein said composition further comprises effective amounts of retinoic acid and nerve growth factor to induce the bone marrow stromal cells to increase production of natriuretic peptides.

20. A natriuretic peptide-producing cell comprising an isolated cell that has been genetically modified with a nucleotide sequence encoding the natriuretic peptide.

21. The natriuretic peptide-producing cell of claim 20, wherein said natriuretic peptide comprise an amino acid sequence selected from the group consisting of SEQ ID NO:2, SEQ ID NO:4, SEQ ID NO:6, SEQ ID NO:18, SEQ ID NO:19, SEQ ID NO:20, and SEQ ID NO:21, or a mammalian homolog of any of the foregoing.

22. The natriuretic peptide-producing cell of claim 20, wherein said nucleotide sequence comprises a sequence selected from the group consisting of SEQ ID NO:1, SEQ ID NO:3, SEQ ID NO:5, SEQ ID NO:10, SEQ ID NO:11, SEQ ID NO:12, SEQ ID NO:13, SEQ ID NO:14, SEQ ID NO:15, SEQ ID NO:16, and SEQ ID NO:17, or a mammalian homolog of any of the foregoing.


SEQUENCE LISTING (21 sequences)

SEQ ID NO: 1 - DNA, 1922 bp, Homo sapiens
ctgtgagatcaccccgtgctcccagcgctcacgtcggtcctcggaaagccggggtcctccctgccttttccagcaacggtggggtggggaggcaggaagaaagcgccaacctaggaccccggagatttgcagcaaaggaagaagcgggagacgggcacttgtctgtgtctccagcgcgttcctgccccccgccgacccggcccatttctatacaaggtcgctctgcccggtctccacctcccacgtgcaggccgcggaggggctcattcccgggccctgatctcagaggcccggaatgtggctgataaatcagagactagacctgcatggcaggcaggcccgacactcagctccaggataaaaggccacggtgtcccgaggagccaggaggagcaccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggtgagagcggagggcagctcagggggattggacagcagcaatgaaagggtcctcacctgctgtcccaagaggccctcatctttcctttggaattagtgataaaggaatcagaaaatggagagactgggtgccctgaccctgtacccaaggcagtcggttcacttgggtgccatgaagggctggtgagccaggggtgggtccctgaggcttggacgcccccattcattgcaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaaggtaagcaccccctgccaccccggccgccttcccccattccagtgtgtgacactgttagagtcactttggggtttgttgtctctgggaaccacactctttgagaaaaggtcacctggacatcgcttcctcttgttaacagccttcagggccaaggggtgcctttgtggaattagtaaatgtgggcttatttcattaccatgcccacaataccttctccccacctcctacttcttatcaaaggggcagaatctcctttgggggtctgtttatcatttggcagccccccagtggtgcagaaagagaaccaaacatttcctcctggtttcctctaaactgtctatagtctcaaaggcagagagcaggatcaccagagcaatgataatccccaatttacagatgaggaaactgaggctcagagagttgcattaagcctcaaacgtctgatgactaacagggtggtgggtggcacacgatgaggtaagctcagcccctgcctccatctcccaccctaaccatcatcaccctctctctttccctgacagtgctgaggcggcattaagaggaagtcctggctgcagacacctgcttctgattccacaaggggctttttcctcaaccctgtggccgcctttgaagtgactcattttttttaatgtatttatgtatttatttgattgttttatataagatggtttcttacctttgagcacaaaatttccacggtgaaataaagtcaacattataagctttatcttttgaaactgatttgtcttggcgcattaaaaataatccctcatttcaaagaa

SEQ ID NO: 2 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - DNA, 845 bp, Homo sapiens
tggcgagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 4 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 5 - DNA, 1803 bp, Canis sp.
cgatcagggatgttggggcggaggaaacggagggaaggagggagcggaggaggcccgaggactgttggtgtccccctcctgcccttttggggccaggcccacttctatacaaggcctgctctccagcctccaccccggcgggtatggtgcaggcgcggaggggcgcattcccccgccctgagctcagcggccggaatgcggccgataaatcagagataaccccaggcgcgggataagggataaaaagcccccgttgccgcgggatccaggagagcacccgcgccccaagcggtgacactcgaccccggtcgcagcgcagcagctcagcagccggacgtctctttccccacttctctccagcgacatggagccctgcgcagcgctgccccgggccctcctgctcctcctgttcttgcacctgtcgccactcggaggccgcccccacccgctgggcggccgcagccccgcctcggaagcctcggaagcctcagaagcctcggggttgtgggccgtgcaggtgagcgctcagcctgcctgaaggccgcggcgggtggcagcaggtcacgggggcttagccactgtcccaagtcctcagtctcccttgggaattagtgataagggaatcagaaagtgacgagattgggtgccaggactccatacccaaggcggcggcttcacttgggtgcaagggtggttccgccccggcgtgggttcctgaggctcaggccgtccattgcaggagctgctgggccgtctgaaggacgcagtttcagagctgcaggcagagcagttggccctggaacccctgcaccggagccacagccccgcagaagccccggaggccggaggaacgccccgtggggtccttgcaccccatgacagtgtcctccaggccctgagaagactacgcagccccaagatgatgcacaagtcagggtgctttggccggaggctggaccggatcggctccctcagtggcctgggctgcaatggtaagccgcctccctgccgccttggctccccctccccagccccctgggttcgacccttggaaccccttctgggtttgttgtctcgggggatcacactctgaggaaaggacatctggacatcgctccttcttgctgacagtcctaagggccaaggagtacgtttctggaaatactacgtgtggacatcgttgtccagggtccctacccacctcctagccccctcctgcctctcgcacccaagggcagaatcatcttaggatggaatcagtcgttgtctggaagcatctccttggagcagaaagagtcctaaacatcgtcctcgtagctctctctgtctgtctgtagccacgaaggcagaggtcagggtcaccagggcagtgatgattcccagttaacagaggaggagactgaggtctagagagatggattattccaaagcctcaaacatccagatcggctgagggtggggttggtggcagggatggctcctgggcttgggaagctcggatcctgcctcagtctcccacctgacgccatcatccccctctctctcctcccacagtgctgagaaagtattaaggaggaagtcccgactgcccacatctgcattggattcttcagcagcccctgagccccttggaagcagatcttatttattcgtatttatttatttatttatttcgattgttttatataagatgatcctgacgcccgagcacggattttccacggtgaaataaagtcaaccttagagcttcttttgaaaccgatttgtccctgtgcattaaaagtaacacatcatttaaaaaaa

SEQ ID NO: 6 - protein, 140 aa, Canis sp.
MEPCAALPRALLLLLFLHLSPLGGRPHPLGGRSPASEASEASEASGLWAVQELLGRLKDAVSELQAEQLALEPLHRSHSPAEAPEAGGTPRGVLAPHDSVLQALRRLRSPKMMHKSGCFGRRLDRIGSLSGLGCNVLRKY

SEQ ID NO: 7 - DNA, 15 bp, Homo sapiens
cgctgctcctgtaac

SEQ ID NO: 8 - DNA, 19 bp, Homo sapiens
gcctcggacttggaaacgt

SEQ ID NO: 9 - DNA, 19 bp, Homo sapiens
tgcagctccgacagtttgc

SEQ ID NO: 10 - DNA, 405 bp, Homo sapiens
atggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaa

SEQ ID NO: 11 - DNA, 456 bp, Homo sapiens
atgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactga

SEQ ID NO: 12 - DNA, 1432 bp, Mus musculus
gaattctcaggtcctgagctcagccggcaggaatgcagctgataaatcagagataaccccacccctactccgtgaaaaggtctggccggacactcagccccagtataaaaggcagaggcaccgttgttgaagacaccagtgcacaagctgcttggggaggcgagacaagggagaacacggcatcattgcctggcccatcgcttctgcggcatggatctcctgaaggtgctgtcccagatgattctgtttctgcttttcctttatctgtcaccgctgggaggtcactcccatcctctgggaagtcctagccagtctccagagcaattcaagatgcaggtgagcactgagggtctgcctgaagggtttgggaagcggcaatgaaaagacctcgagtcctttgggaattagccatgtgagagtcagcaaagtgaaagattgggcagcatatctcttaactgatgagcactatggaaggatgggggattcaggtgtgtgtgtttctgacgtctgggctccccaatccatcacagaagctgctggagctgataagagaaaagtcagaggaaatggcccagagacagctcttgaaggaccaaggcctcacaaaagaacacctaaaaagagtccttcggtctcaaggcagcaccctccgggtccagcagagacctcaaaattccaaggtgacacatatctcaagctgctttgggcacaagatagaccggatcggatccgtcagtcgtttgggctgtaacggtgagcacctaccttgccacttccctgcaaagctgcaccacccatcccatccccgtgcatgctacccttagaggcccctaggtttgctatctggcatactcctgcagcctgtcaggaaatatcacatgggttctgcattacattctcacaggtcagcacctaccttccatcagagggtcacacgctctgaggagcagactgctgatgtctatcaccccttcacaaggcagaaagagtctgagcattcccctcaggcaaagggcatgcccaacccactttacaggagaaacagaggccctctgagatagctttttccagagccttaaacttcgacatcatctggggactgaagatgggggtgtggtggtggtgggggactcggcacctgcttcagtttcacttcgagtgtgacattgcctgtctctcctccacagcactgaagttgttgtaggaagacctccttggctgcaggagagctccagtttctgactctgccgggtctctttccctagctctggaccacctctgaagtgatcctatttatttatttatttatatttattatttatttttatttttattttttaatttaattttgttgtttttcacagctgttgttacttggagcacaaactgccacaacataataaacatacgttatttcctgcttttgaaaagg

SEQ ID NO: 13 - DNA, 1468 bp, Mus musculus
gaattctcaggtcctgagctcagccggcaggaatcagctgataaatcagagataaccccacccctactccgtgaaaaggtctggccggacactcagccccagtataaaaggcagaggcaccgttgttgaagacaccagtgcacaagctgcttggggaggcgagacaagggagaacacggcatcattgcctggcccatcgcttctgcggcatggatctcctgaaggtgctgtcccagatgattctgtttctgcttttcctttatctgtcaccgctgggaggtcactcctatcctctgggaagtcctagccagtctccagagcaattcaagatgcaggtgagcactgagggtctgcctgaagggtttgggaagcggcaatgaaaagacctcgagtcctttgggaattagccatgtgagagtcagcaaactgaaagattgggcagcatatctcttaactgatgagcactatggaaggatgggggattcaggtgtgtgtgtttctgacgtctgggctccccaatccatcacagaagctgctggagctgataagagaaaagtcggaggaaatggcccagagacagctcttgaaggaccaaggcctcacaaaagaacacccaaaaagagtccttcggtctcaaggcagcaccctccgggtccagcagagacctcaaaattccaaggtgacacatatctcaagctgctttgggcacaagatagaccggatcggatccgtcagtcgtttgggctgtaacggtgagcacctaccttgccacttccctgcaaagctgcacacccatcccatccccgtgcatgctacccttagaggcccctaggtttgctatctggcatactcctgcagcctgtcaggaaatatcacatgggttctgcattacattctcacaggtcagcacctaccttccatcagaggggtcacacgctctgagggagcagactgcctgatgtctaatcaccccttcacaaggcagaaagagttctgagcatttcccctcaggcaaagggcatgcccaacccactttacaggagaaacagaggccctgtgagatagctttttccagagccttaaacttcgacatcatctggggactgaagatgggggtgtggtggtggtgggggactcggcacctgcttcagtttcacttccgagtgtgacattgccctgtctctcctccccacagcactgaagttgttgtaggaagacctcctggctgcaggagactccagtttctgactctgcctgggtctctttccccagctctgggaccacctttgaagtgatcctatttatttatttatttatatttatttttatttttattttttaatttattttgttgtttttctacaagactgtttcttatcttggagcacaaacttgccacaacataataaacatagcgtatttcctgcttttgaaaaggatttgtgtccgtgagtttcaatctatctct

SEQ ID NO: 14 - DNA, 628 bp, Rattus norvegicus
gcgagacaagagagagcaggacaccatcgcagctgcctggcccatcacttctgcagcatggatctccagaaggtgctgccccagatgattctgctcctgcttttccttaatctgtcgccgctgggaggtcactcccatcccctgggaagtcctagccagtctccagaacaatccacgatgcagaagctgctggagctgataagagaaaagtcagaggaaatggctcagagacagctctcaaaggaccaaggccctacaaaagaacttctaaaaagagtccttaggtctcaagacagcgccttccggatccaggagagacttcgaaattccaagatggcacatagttcaagctgctttgggcagaagatagaccggatcggcgcagtcagtcgcttgggctgtgacgggctgaggttgttttaggaagacctcctggctgcagactccggcttctgactctgcctgcggctcttctttccccagctctgggaccacctctcaagtgatcctgtttatttatttgtttatttatttatttttatgttgctgattttctacaagactgtttcttatcttccagcacaaacttgccacagtgtaataaacatagcctatttcttgcttttgg

SEQ ID NO: 15 - DNA, 1479 bp, Sus scrofa
caggctgctaggaagtgaaaagtgaacctggacccagctcagcggcagcagcagcggcagcaggcagcagcctctatcctctcctccagccacatgggcccccggatggcgcttccccgcgtgctcctgctcctgttcttgcacctgttgctgctaggatgccgttcccatccactgggtggcgctggcctggcctcagaactgccagggatacaggtgagccctgatgaactgcttagacttggttggctgggagggcgcggacagcagcaactaacgggtccccacctactgttccaagagggctctaacctcctttgggaactagtgataaggggttagaaggcagccaggctgggggtgaggaccccgctcccaaggcagttggttcgcttcagcaccatcaagagtgatgggtccaggtgcgagttcctgaggctcgggctcccccacccatcccaggagctgctggaccgcctgcgagacagggtctccgagctgcaggcggagcggacggacctggagcccctccggcaggaccgtggcctcacagaagcctgggaggcgagggaagcagcccccacgggggttcttgggccccgcagtagcatcttccaagtcctccggggaatacgcagccccaagacgatgcgtgactctggctgctttgggcggaggctggaccggatcggctccctcagcggcctgggctgcaatggtgagcacccacccccattcccactgcacgccccggttagcatcacttctgggtttgatgtctctggggaccaaactccgagaaaaggacacctggatatcactctttcttgttgccagtcctcaaggccaaggagcgccttcctggaaaaattaaatttggacagcattcactagcatgactatgagtccccacccaccttctcgccaccccctgcctctctcacccaaggcggcagaattactttaggatgtaaattctgtcattgcctggctgccgctcctgggagcaaaaagagaactaaacctcttccccctggtttcccctcaactgtctgtggctgcaaaggcagagggcaggatcaccagggtgatgacaagtcccagcttacaaggaggaaactcaggtccagagagatggattatcccaaagccccaaacatccagttctgctgaagaaggcgggtggcaggggtggcacgtggtggggggaagcccaggtcctgcctgcctctcaccctaatgtcatcctcaccctctctctcccccccacagtgctcaggaggtactgagaagtcctggctgacaacctctgtgtccgcttctccaacgcccctcccctgctccccttcaaagcaactcctgtttttatttatgtatttatttatttatttatttggtggttgtatataagacggttcttatttgtgagcacattttttccatggtgaaataaagtcaacattagagctctgtcttttg

SEQ ID NO: 16 - DNA, 1769 bp, Bos taurus
ctgcagctgagggtcctgggggttgtcggggctgctcaaggcagaggggctgtgacaagcaggctggactgataactttaaaagggcatcttctgctgcttcctcactcagctgctttatcactgcaagtgacagaatggggagggttccgtccctctcccggacgagctcccagagagccagggggctataaaaagaggaggctcagggcagctgggagacagagacggacaaaggccaacagcaaaaggccaaagaggacagggaggaggcagcaagcaccagaccgaccattccttgaccgacgccagcatgggctcctccgccatcaccgtgagcttcctcctctttctggcatttcagctcccagggcaaacaggagcaaatcccgtgtatggctctgtgtccaatgcagacctgatggatttcaaggtagggccagggaacggcgatggtctggggctgagggggttgtgacattgtgccaggcgagcgagacctctccctttccctgttttccttttgtaaagaatttgctggaccgtttggaggacaagatgcctttagaagatgaggctgtgccctcacaagtactaagtgagcagaatgaagaagctggggcccctctcagccccctttcagagatgcctccctggatgggggaggtcaacccagcccagagagaggggggcgtcctcgggcggggcccctgggaatcctccgatagatctgccctcctgaagagcaagctgagggcactgctcactgcccctcggagcctgcggaggtccagctgcttcgggggaaggatggacaggattggagcccagagtggattgggctgcaacagcttccgggtaagaggacctgagaatggaaatgggatggggaggaaggaaattgtggcttcattgaagttcaaaccttgtgaaagaacatcgccagggaatgccttcagtaggaaagggacagcatagaagcaacccctttgaaatttctgccccaacttggcagggaggagggtgtgctctgagtctcaggacaatgataccaacctagctacagttttctgagagaatgctaagaaaaaaagactttactgccacgagcactggggacttaaattgttcatggggccaaataacctgtgctttgctgattggtagtttgtgtcctttgcagaatcatcagatcccaaaggattgaaattgagcaggactgactttactagttcctaatgggcaatttgtttaccagtttatagaagtcagagggtcatcaggctggagtggaggctggtgggaagggagcacagtctgatgaagctggcttttccagtggagtcaggtcaccaaaccaaacatgtctctgctctcttgtagtatcgaagataatggccagggaggaaaaggcaggccaggccctgggcagtcttcaagagaatcccctggggtctctcactcaactttgtcgcatctggttgccatcaagttgagctgtgaccgagcattcaagcatcagcttcttgtcaacatttctcacattttatgctaaatgtagacaaagtgatttaactgtggccttctccacctctcccacccatgtgttaagttttaatcacctgttaccaacatcagtttgaaaatgaataaacttcagcaccatggacagaagcagtaggctcgggttggtgtgatttctttcatttccggaagggagttcagcctgatactccttgtcattttaccttttgttggagagaagaattc

SEQ ID NO: 17 - DNA, 1301 bp, Rattus norvegicus
gcgagacaagagagagcaggacaccatcgcagctgcctggcccatcacttctgcagcatggatctccagaaggtgctgccccagatgattctgctcctggttttccttaatctgtcgccgctgggaggtcactcccatcccctgggaagtcctagccagtctccagaacaatccacgatgcaggtgagcaccgagggtctgcatagggagatggagcctgcctgaagggttttgggcagcagcaatgaaaagacctcatgtcctttgggaattaaccacgcgagagtcaggaaacggaaagattgggcagcagatcccttaaccacaggcactgtggaagggtgggggagccaggtgtgtatgtgtgtgtgtgtctgaggtctgggcttcccaattcgtcacagaagctgctggagctgataagagaaaagtcagaggaaatggctcagagacagctctcaaaggaccaaggccctacaaaagaacttctaaaaagagtccttaggtctcaagacagcgccttccggatccaggagagacttcgaaattccaagatggcacatagttcaagctgctttgggcagaagatagaccggatcggcgcagtcagtcgcttgggctgtgacggtgagcacctaccttgccgcttccctgcaaagctgcacgcatcccgttcccctgcatgccgccctcagaggccccttggtttgctctcagacatacttgcacagcctgcctctaccttaccgacagtcttcaagaccaaggcagtctgtcaggaagtctcacatgggtacttcattacaccgtcccaggtgagcacctacctccttcagaggtgtcacaggcttcccagggaacagactgcctgatgtctgatcactctgagcatctcccctccgtcttcaccaaactgaattatccgaggcaaagggcaggcccagtgagatagcttttcccagagccgttaaacttcgacatcatctgggaaccaaagatgggggtgcggtgtggcaggggaagctcagctcctgcctcagtttcactccccagtctgacactggtttctcctcccacagggctgaggttgttttaggaagacctcctggctgcagactccggcttctgactctgcctgcggctcttctttccccagctctgggaccacctctcaagtgatcctgtttatttatttgtttatttatttatttttatgttgctgattttctacaagactgtttcttatcttccagcacaaacttgccacagtgtaataaacatagcctatttcttgcttttgg

SEQ ID NO: 18 - protein, 152 aa, Bos taurus
MGSSAITVSFLLFLAFQLPGQTGANPVYGSVSNADLMDFKNLLDRLEDKMPLEDEAVPSQVLSEQNEEAGAPLSPLSEMPPWMGEVNPAQREGGVLGRGPWESSDRSALLKSKLRALLTAPRSLRRSSCFGGRMDRIGAQSGLGCNSFRYRR

SEQ ID NO: 19 - protein, 121 aa, Mus musculus
MDLLKVLSQMILFLLFLYLSPLGGHSYPLGSPSQSPEQFKMQKLLELIREKSEEMAQRQLLKDQGLTKEHPKRVLRSQGSTLRVQQRPQNSKVTHISSCFGHKIDRIGSVSRLGCNALKLL

SEQ ID NO: 20 - protein, 121 aa, Rattus norvegicus
MDLQKVLPQMILLLLFLNLSPLGGHSHPLGSPSQSPEQSTMQKLLELIREKSEEMAQRQLSKDQGPTKELLKRVLRSQDSAFRIQERLRNSKMAHSSSCFGQKIDRIGAVSRLGCDGLRLF

SEQ ID NO: 21 - protein, 131 aa, Sus scrofa
MGPRMALPRVLLLLFLHLLLLGCRSHPLGGAGLASELPGIQELLDRLRDRVSELQAERTDLEPLRQDRGLTEAWEAREAAPTGVLGPRSSIFQVLRGIRSPKTMRDSGCFGRRLDRIGSLSGLGCNVLRRY